(19) United States
(12) Patent Application Publication
(10) Pub. No.: US 2024/0167848 A1
(43) Pub. Date: May 23, 2024

(54) STALK SENSORS AND RELATED DEVICES, SYSTEMS, AND METHODS

(71) Applicant: Ag Leader Technology, Ames, IA (US)

(72) Inventors: Brett Johnson, Ames, IA (US); Stuart Helming, Ames, IA (US); Nathan Risius, Huxley, IA (US); Mel Zayas, Ames, IA (US); Kurt Larsen, Ames, IA (US); Lucas Roe, Ames, IA (US); Jacob Werner, Ames, IA (US); Christian Erwin, Ames, IA (US)

(21) Appl. No.: 18/516,514

(22) Filed: Nov. 21, 2023

Related U.S. Application Data
(60) Provisional application No. 63/427,028, filed on Nov. 21, 2022.

(51) Int. Cl. G01D 5/14; A01D 34/00

(52) U.S. Cl. CPC G01D 5/14; A01D 34/006

(57) ABSTRACT

A stalk sensor comprising a first wand comprising a contact surface at a distal end, a first magnet disposed at a distal end of the first wand, a second wand comprising a contact surface at a distal end, and a second magnet disposed at a distal end of the second wand, the magnet configured to attract the first magnet and thereby the first wand into contact with the second wand at the contact surfaces with the second wand, wherein the first contact surface and the second contact surface are in contact when the stalk sensor is in a home position. The stalk sensor wherein the wands are self-aligning on horizontal and vertical axes.

### CROSS-REFERENCE TO RELATED APPLICATION(S)

This application claims the benefit under 35 U.S.C. § 119(e) to U.S. Provisional Application 63/427,028, filed Nov. 21, 2022, and entitled Stalk Sensors and Associated Devices, Systems and Methods, which is hereby incorporated herein by reference in its entirety for all purposes.

### TECHNICAL FIELD

The disclosure relates to agricultural sensors and implements, more particularly to stalk sensors for use on harvesters or other agricultural implements.

### BACKGROUND

Various sensors for measuring and counting stalks and collecting certain data relating to harvest and planting are known in the art. Various sensors are disclosed in U.S. application Ser. No. 16/445,161, U.S. application Ser. No. 16/800,469, U.S. application Ser. No. 17/013,037, and U.S. application Ser. No. 17/226,002, each of which has been incorporated by reference herein.

### BRIEF SUMMARY

Disclosed herein are various magnetic tip wands configured to bring the wand(s) to a resting position between stalk impacts. In these implementations, the amount the wand(s) may bounce off the stalk is reduced or eliminated such that the sensor signal is clearer to better distinguish between stalks and areas of noise. This bouncing may create a false peak in the signal that the system identifies as a stalk, leading to inaccuracies in the data. Further, various of these implementations may allow for small stalks to be more distinguishable from leaves, weeds, or other debris.

In Example 1, a stalk sensor comprising: a first wand comprising a contact surface at a distal end; a first magnet disposed at a distal end of the first wand; and a second wand comprising a contact surface at a distal end, wherein the first contact surface and the second contact surface are in contact when the stalk sensor is in a home position.

Example 2 relates to the stalk sensor of any of Examples 1 and 3-14, further comprising a ferrous material disposed at a distal end of the second wand, the ferrous material configured to attract the first magnet and thereby the first wand into contact with the second wand at the contact surfaces.

Example 3 relates to the stalk sensor of any of Examples 1-2 and 4-14, wherein the first wand and second wand are self-aligning along the horizontal axis.

Example 4 relates to the stalk sensor of any of Examples 1-3 and 5-14, further comprising a second magnet disposed at a distal end of the second wand, the magnet configured to attract the first magnet and thereby the first wand into contact with the second wand at the contact surfaces with the second wand.

Example 5 relates to the stalk sensor of any of Examples 1-4 and 6-14, wherein a north pole of the first magnet is adjacent to the contact surface of the first wand and wherein a south pole of the second magnet is adjacent to the contact surface of the second wand.

Example 6 relates to the stalk sensor of any of Examples 1-5 and 7-14, wherein north and south poles of the first magnet and second magnet are each adjacent to the contact surfaces.

Example 7 relates to the stalk sensor of any of Examples 1-6 and 8-14, wherein poles of the first magnet and second magnet are oriented perpendicular to a direction of deflection of the first wand and second wand.

Example 8 relates to the stalk sensor of any of Examples 1-7 and 9-14, wherein the wands are self-aligning on horizontal and vertical axes.

Example 9 relates to the stalk sensor of any of Examples 1-8 and 10-14, further comprising a hinge shield configured to extend beyond a hinged portion of the first wand and restrict deflection of the first wand.

Example 10 relates to the stalk sensor of any of Examples 1-9 and 11-14, further comprising a third magnet at a distal end of the hinge shield configured to attract a sensing magnet on the first wand and return the stalk sensor the home position.

Example 11 relates to the stalk sensor of any of Examples 1-10 and 12-14, further comprising a third magnet at a distal end of the hinge shield configured to repel a sensing magnet on the first wand and dampen movement of the first wand toward the home position.

Example 12 relates to the stalk sensor of any of Examples 1-11 and 13-14, wherein the hinge shield is configured to be mounted to a harvester row unit.

Example 13 relates to the stalk sensor of any of Examples 1-12 and 14, wherein the first wand and second wand are configured to flex and separate the first contact surface and the second contact surface as a stalk passes through the stalk sensor.

Example 14 relates to the stalk sensor of any of Examples 1-13, wherein the stalk sensor is configured to measure deflection of the first wand and second wand to count stalks as stalks pass through the sensor.

In Example 15, a sensing wand comprising a magnet at a distal end of the wand wherein a first pole of the magnet faces a contact surface at the distal end of the wand and a second pole of the magnet if faced away from the contact surface.

Example 16 relates to the sensing wand of any of Examples 15 and 17, wherein a second sensing wand comprises a ferrous metal at a distal end of the second sensing wand configured to attract the magnet of the sensing wand.

Example 17 relates to the sensing wand of any of Examples 15-16, further comprising a hinge shield configured to extend beyond a hinged portion of the sensing wand and restrict deflection of the sensing wand.

In Example 18, a sensing wand comprising a magnet at the distal end of the wand wherein north and south poles of the magnet are oriented perpendicular to a direction of deflection of the sensing wand.

Example 19 relates to the sensing wand of any of Examples 18 and 20, further comprising a hinge shield configured to extend beyond a hinged portion of the sensing wand and restrict deflection of the sensing wand.

Example 20 relates to the sensing wand of any of Examples 18-19, wherein the hinge shield is configured to be mounted to a harvester row unit.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a top view of sensor wands with a single magnet, according to one implementation.

FIG. 2 is a top view of sensor wands in a dual magnet configuration, according to one implementation.

FIG. 3 is a perspective view of a stalk sensor in a dual magnet configuration, according to one implementation.

FIG. 4 is a perspective view of sensor wands in a perpendicular dual magnet configuration, according to one implementation.

FIG. 5 is a perspective view of a single wand with a perpendicular magnet, according to one implementation.

FIG. 6 is a perspective view of a single wand with a perpendicular magnet, according to one implementation.

FIG. 7 is a perspective view of a single wand with two magnets in a parallel orientation with opposite poles abutting the contact surface of the wand, according to one implementation.

FIG. 8 is a graph showing sensor signals over time for a stalk sensor without magnets, according to one implementation.

FIG. 9 is a graph showing sensor signals over time for a stalk sensor in a dual magnet configuration, according to one implementation.

FIG. 10 is a graph showing reduced signal noise for sensor signals over time for stalk sensors in a dual magnet configuration, according to one implementation.

FIG. 11 is a graph showing reduced signal noise for sensor signals over time for stalk sensors in a dual magnet configuration, according to one implementation.

FIG. 12 is a schematic depiction of a stalk sensor in a dual magnet configuration, according to one implementation.

FIG. 13 is a cross-sectional side view of a sensor wand with a hinge shield, according to one implementation.

FIG. 14 is a perspective view of a sensor wand with a hinge shield, according to one implementation.

FIG. 15 is a cross-sectional side view of a sensor wand with a hinge shield and an attracting magnet, according to one implementation.

FIG. 16 is a cross-sectional side view of a sensor wand with a hinge shield and an opposing magnet, according to one implementation.

### DETAILED DESCRIPTION

Described herein are hinged/flexible wand(s) for use in conjunction with a sensor configured to count corn stalks. Various sensors and wands for counting corn stalks are described in U.S. patent application Ser. No. 17/013,037, which is incorporated herein by reference. The herein disclosed implementations of the wand(s) include a magnet configured to reduce vibration and rebounding by returning and securing the wand(s) to a home/rest position. In various implementations, the disclosed wand implementations are self-aligning.

As would be appreciated, stalk sensor may include flexible wands. The wands are flexible elongate members that deflect when stalks pass through the sensor. The deflection is measured by the sensor to generate a signal which may then be interpreted to count and sense stalks. Optionally, the deflection is measure by a magnetic sensor measuring the magnetic field of a sensing magnet located on the wands. Various stalk sensor designs, methods, and system are described in the incorporated references.

Certain of the disclosed implementations can be used in conjunction with any of the devices, systems or methods taught or otherwise disclosed in U.S. Pat. No. 10,684,305 issued Jun. 16, 2020, entitled “Apparatus, Systems and Methods for Cross Track Error Calculation From Active Sensors,” U.S. patent application Ser. No. 16/121,065, filed Sep. 4, 2018, entitled “Planter Down Pressure and Uplift Devices, Systems, and Associated Methods,” U.S. Pat. No. 10,743,460, issued Aug. 18, 2020, entitled “Controlled Air Pulse Metering apparatus for an Agricultural Planter and Related Systems and Methods,” U.S. Pat. No. 11,277,961, issued Mar. 22, 2022, entitled “Seed Spacing Device for an Agricultural Planter and Related Systems and Methods,” U.S. patent application Ser. No. 16/142,522, filed Sep. 26, 2018, entitled “Planter Downforce and Uplift Monitoring and Control Feedback Devices, Systems and Associated Methods,” U.S. Pat. No. 11,064,653, issued Jul. 20, 2021, entitled “Agricultural Systems Having Stalk Sensors and/or Data Visualization Systems and Related Devices and Methods,” U.S. Pat. No. 11,297,768, issued Apr. 12, 2022, entitled “Vision Based Stalk Sensors and Associated Systems and Methods,” U.S. patent application Ser. No. 17/013,037, filed Sep. 4, 2020, entitled “Apparatus, Systems and Methods for Stalk Sensing,” U.S. patent application Ser. No. 17/226,002 filed Apr. 8, 2021, and entitled “Apparatus, Systems and Methods for Stalk Sensing,” U.S. Pat. No. 10,813,281, issued Oct. 27, 2020, entitled “Apparatus, Systems, and Methods for Applying Fluid,” U.S. patent application Ser. No. 16/371,815, filed Apr. 1, 2019, entitled “Devices, Systems, and Methods for Seed Trench Protection,” U.S. patent application Ser. No. 16/523,343, filed Jul. 26, 2019, entitled “Closing Wheel Downforce Adjustment Devices, Systems, and Methods,” U.S. patent application Ser. No. 16/670,692, filed Oct. 31, 2019, entitled “Soil Sensing Control Devices, Systems, and Associated Methods,” U.S. patent application Ser. No. 16/684,877, filed Nov. 15, 2019, entitled “On-The-Go Organic Matter Sensor and Associated Systems and Methods,” U.S. Pat. No. 11,523,554, issued Dec. 13, 2022, entitled “Dual Seed Meter and Related Systems and Methods,” U.S. patent application Ser. No. 16/891,812, filed Jun. 3, 2020, entitled “Apparatus, Systems and Methods for Row Cleaner Depth Adjustment On-The-Go,” U.S. Pat. No. 11,678,607, issued Jun. 20, 2023, entitled “Apparatus, Systems, and Methods for Eliminating Cross-Track Error,” U.S. patent application Ser. No. 16/921,828, filed Jul. 6, 2020, entitled “Apparatus, Systems and Methods for Automatic Steering Guidance and Visualization of Guidance Paths,” U.S. patent application Ser. No. 16/939,785, filed Jul. 27, 2020, entitled “Apparatus, Systems and Methods for Automated Navigation of Agricultural Equipment,” U.S. patent application Ser. No. 16/997,361, filed Aug. 19, 2020, entitled “Apparatus, Systems and Methods for Steerable Toolbars,” U.S. Pat. No. 11,785,881, issued Oct. 17, 2023, entitled “Adjustable Seed Meter and Related Systems and Methods,” U.S. patent application Ser. No. 17/011,737, filed Sep. 3, 2020, entitled “Planter Row Unit and Associated Systems and Methods,” U.S. patent application Ser. No. 17/060,844, filed Oct. 1, 2020, entitled “Agricultural Vacuum and Electrical Generator Devices, Systems, and Methods,” U.S. patent application Ser. No. 17/105,437, filed Nov. 25, 2020, entitled “Devices, Systems and Methods For Seed Trench Monitoring and Closing,” U.S. patent application Ser. No. 17/127,812, filed Dec. 18, 2020, entitled “Seed Meter Controller and Associated Devices, Systems and Methods,” U.S. patent application Ser. No. 17/132,152, filed Dec. 23, 2020, entitled “Use of Aerial Imagery For Vehicle Path Guidance and Associated Devices, Systems, and Methods,” U.S. patent application Ser. No. 17/164,213, filed Feb. 1, 2021, entitled “Row Unit Arm Sensor and Associated Systems and Methods,” U.S. patent application Ser. No. 17/170,752, filed Feb. 8, 2021, entitled “Planter Obstruction Monitoring and Associated Devices and Methods,” U.S. patent application Ser. No. 17/225,586, filed Apr. 8, 2021, entitled “Devices, Systems, and Methods for Corn Headers,” U.S. Pat. No. 11,758,848, issued Sep. 19, 2023, entitled “Devices, Systems, and Methods for Sensing the Cross Sectional Area of Stalks,” U.S. patent application Ser. No. 17/323,649, filed May 18, 2021, entitled “Assisted Steering Apparatus and Associated Systems and Methods,” U.S. patent application Ser. No. 17/369,876, filed Jul. 7, 2021, entitled “Apparatus, Systems, and Methods for Grain Cart-Grain Truck Alignment and Control Using GNSS and/or Distance Sensors,” U.S. patent application Ser. No. 17/381,900, filed Jul. 21, 2021, entitled “Visual Boundary Segmentations and Obstacle Mapping for Agricultural Vehicles,” U.S. patent application Ser. No. 17/461,839, filed Aug. 30, 2021, entitled “Automated Agricultural Implement Orientation Adjustment System and Related Devices and Methods,” U.S. patent application Ser. No. 17/468,535, filed Sep. 7, 2021, entitled “Apparatus, Systems, and Methods for Row-by-Row Control of a Harvester,” U.S. patent application Ser. No. 17/526,947, filed Nov. 15, 2021, entitled “Agricultural High Speed Row Unit,” U.S. patent application Ser. No. 17/566,678, filed Dec. 20, 2021, entitled “Devices, Systems, and Method For Seed Delivery Control,” U.S. patent application Ser. No. 17/576,463, filed Jan. 14, 2022, entitled “Apparatus, Systems, and Methods for Row Crop Headers,” U.S. patent application Ser. No. 17/724,120, filed Apr. 19, 2022, entitled “Automatic Steering Systems and Methods,” U.S. patent application Ser. No. 17/742,373, filed May 11, 2022, entitled “Calibration Adjustment for Automatic Steering Systems,” U.S. patent application Ser. No. 17/902,366, filed Sep. 2, 2022, entitled “Tile Installation System with Force Sensor and Related Devices and Methods,” U.S. patent application Ser. No. 17/939,779, filed Sep. 7, 2022, entitled “Row-by-Row Estimation System and Related Devices and Methods,” U.S. patent application Ser. No. 18/215,721, filed Jun. 28, 2023, entitled “Seed Tube Guard and Associated Systems and Methods of Use,” U.S. patent application Ser. No. 18/087,413, filed Dec. 22, 2022, entitled “Data Visualization and Analysis for Harvest Stand Counter and Related Systems and Methods,” U.S. patent application Ser. No. 18/097,804, filed Jan. 17, 2023, entitled “Agricultural Mapping and Related Systems and Methods,” U.S. patent application Ser. No. 18/101,394, filed Jan. 25, 2023, entitled “Seed Meter with Integral Mounting Method for Row Crop Planter and Associated Systems and Methods,” U.S. patent application Ser. No. 18/102,022, filed Jan. 26, 2023, entitled “Load Cell Backing Plate and Associated Devices, Systems, and Methods,” U.S. patent application Ser. No. 18/116,714, filed Mar. 2, 2023, entitled “Cross Track Error Sensor and Related Devices, Systems, and Methods,” U.S. patent application Ser. No. 18/203,206, filed May 30, 2023, entitled “Seed Tube Camera and Related Devices, Systems and Methods,” U.S. patent application Ser. No. 18/209,331, filed Jun. 13, 2023, entitled “Apparatus, Systems and Methods for Image Plant Counting,” U.S. patent application Ser. No. 18/217,216, filed Jun. 30, 2023, entitled “Combine Unloading On-The-Go with Bin Level Sharing and Associated Devices, Systems, and Methods,” U.S. patent application Ser. No. 18/229,974, filed Aug. 3, 2023, entitled “Hydraulic Cylinder Position Control for Lifting and Lowering Towed Implements,” U.S. patent application Ser. No. 18/230,534, filed Aug. 4, 2023, entitled “Single-Step Seed Placement in Furrow and Related Devices, Systems, and Methods,” U.S. patent application Ser. No. 18/238,334, filed Aug. 25, 2023, entitled “Combine Yield Monitor Automatic Calibration System and Associated Devices and Methods,” U.S. patent application Ser. No. 18/367,929, filed Sep. 13, 2023, entitled “Hopper Lid with Magnet Retention and Related Systems and Methods,” U.S. Patent Application 63/445,960, filed Feb. 15, 2023, entitled “Ear Shelling Detection and Related Devices, Systems, and Methods,” U.S. Patent Application 63/445,550, filed Feb. 14, 2023, entitled “Liquid Flow Meter and Flow Balancer,” U.S. Patent Application 63/466,144, filed May 12, 2023, entitled “Devices, Systems, and Methods for Providing Yield Maps,” U.S. Patent Application 63/466,560, filed May 15, 2023, entitled “Devices, Systems, and Methods for Agricultural Guidance and Navigation,” U.S. Patent Application 63/524,065, filed Jun. 29, 2023, entitled “Ring Assembly,” U.S. Patent Application 63/525,525, filed Jul. 7, 2023, entitled “Assisted Steering Systems and Associated Devices and Methods for Agricultural Vehicles,” U.S. Patent Application 63/593,837, filed Oct. 27, 2023, entitled “Agricultural Implement Position Sensor and Related Devices, Systems, and Methods,” each of which is incorporated herein by reference.

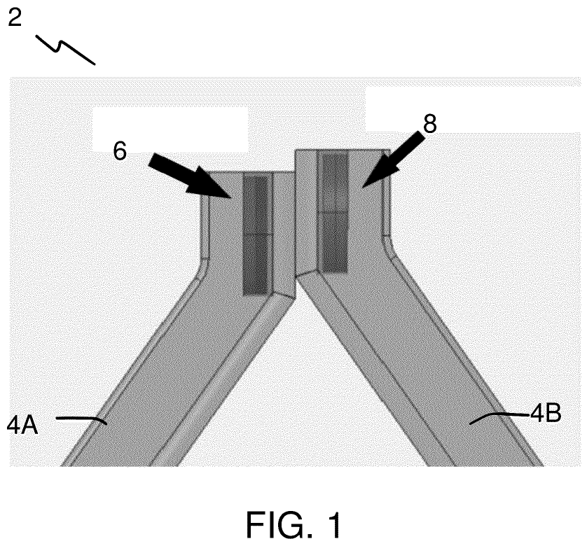

As can be seen in FIG. 1, a stalk sensor 2 includes one wand 4A that includes a magnet 6 and a second wand 4B that includes a ferrous material 8 at their respective ends such that the magnet 6 is attracted to the ferrous material 8 causing the tips of the wands 4A, 4B to come into contact. Yet, as can be seen, in these implementations, the tips of the wands 4A, 4B may become misaligned because the magnet 6 is only generally attracted to the ferrous material 8 and nothing provides alignment.

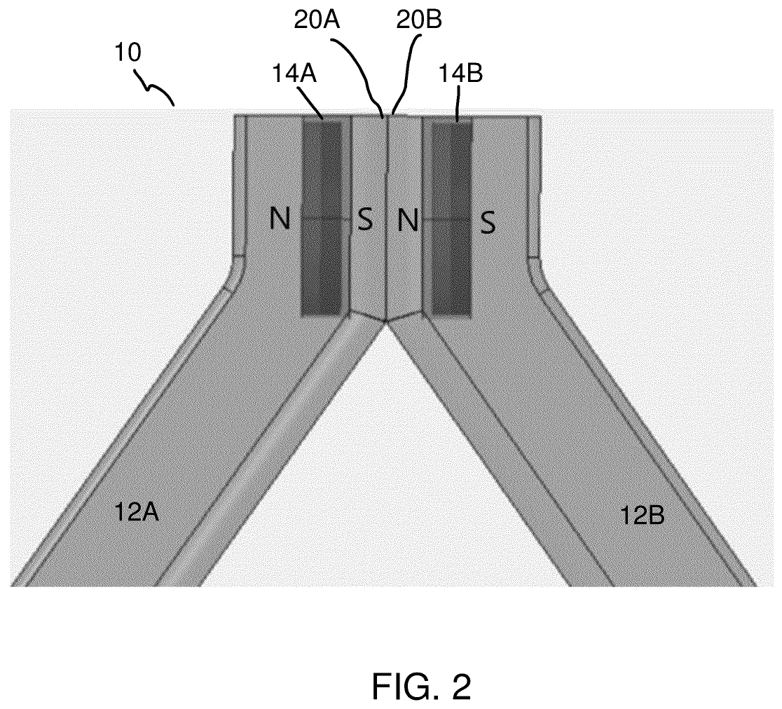

FIG. 2 shows a stalk sensor 10 implementation having two magnets 14A, 14B one in each respective wand 12A, 12B tip. In these implementations, the magnets 14A, 14B are orientated within the wands 12A, 12B such that opposite poles of the magnets 14A, 14B face each other and the tips of the wands 12A, 12B are thereby attracted to each other. That is, when separated the wands 12A 12B will rebound towards one another at the location of the magnets 14A, 14B by magnetic forces.

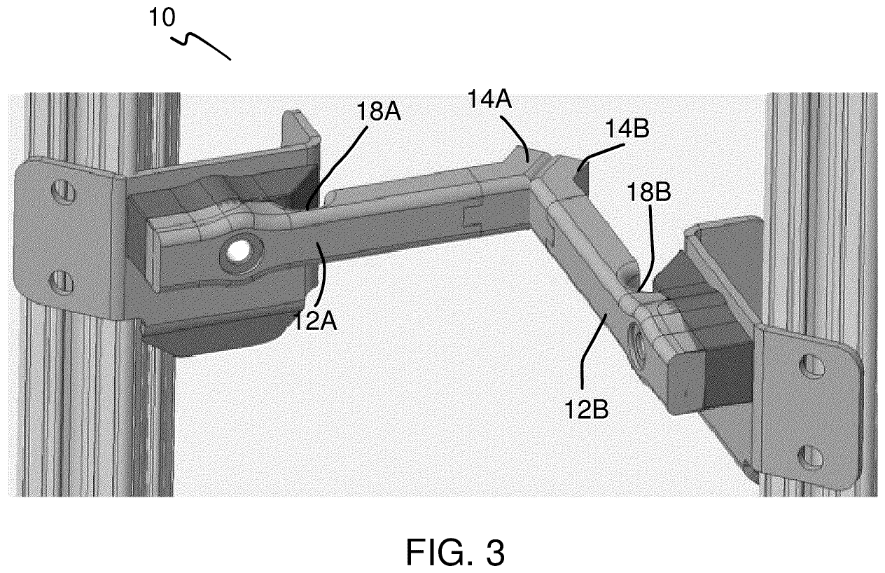

FIG. 3 shows a stalk sensor 10 implementation with self-aligning wands 12A, 12B from a perspective view at the home/rest position. When at the home position, the wand 12A, 12B tips are in contact and held there by magnetic forces of the magnets 14A, 14B embedded within the wands 12A, 12B. As stalks pass through the sensor the wands 12A, 12B flex at the hinge points 18A, 18B causing the wands 12A, 12B to separate and allow the stalk to pass. The magnetic force of the magnets is overcome by the stalk separating the wands 12A, 12B but once the stalk has passed through the sensor 10 the magnetic forces from the magnets 14A, 14B being the wands 12A, 12B back to the home position.

That is, various implementations include magnets 14A, 14B on or in the contacting faces of the two wands 12A, 12B, with poles oriented on the face to attract to each other north to south (positive to negative). These configurations achieve the attraction and self-aligning due to the characteristics of magnets.

In these and other implementations, the sensor 10 using two magnets 14A, 14B, one in each wand 12A, 12B which allows smaller magnets 14A, 14B to be used when compared to a single magnet sensor (such as that shown in FIG. 1) to generate the same amount of magnetic force.

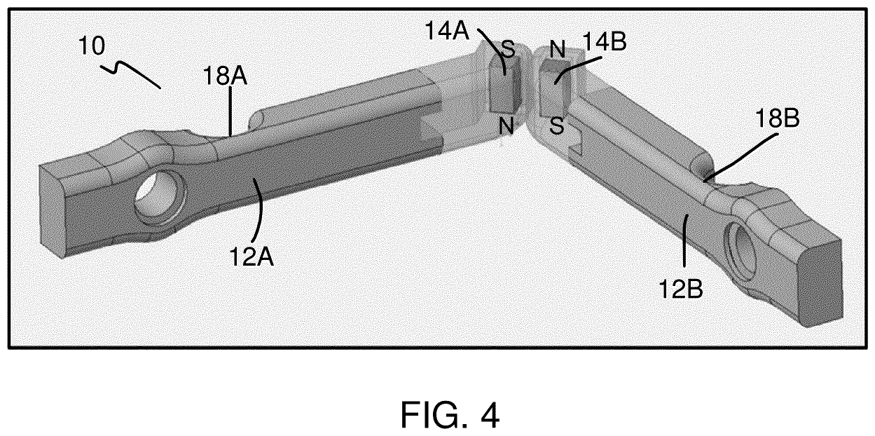

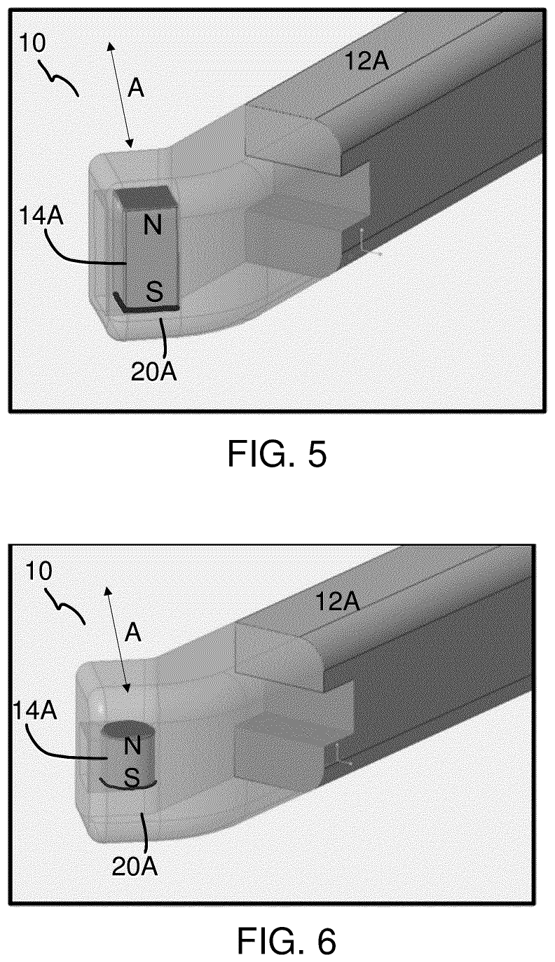

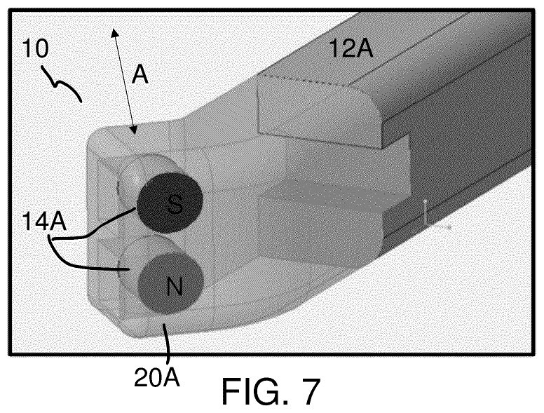

Turning to FIGS. 4-7, in alternative implementations, the magnets 14A, 14B may be arranged within the wands 12A, 12B so that the poles are perpendicular to the direction of motion (shown at arrow A). In these and other implementations, a single wand 12 design using a single magnet 14 can be used interchangeably on the left or right side of the sensor 10, with the wand 12A, 12B simply reversed when placed on the opposite side. The two magnets 14A, 14B arranged vertically so the poles are facing the opposite direction parallel to the direction of motion improves self-aligning in all directions because the poles would be aligned vertically as well as causing horizontal alignment by use of magnets 14A, 14B on the contact faces 20A, 20B.

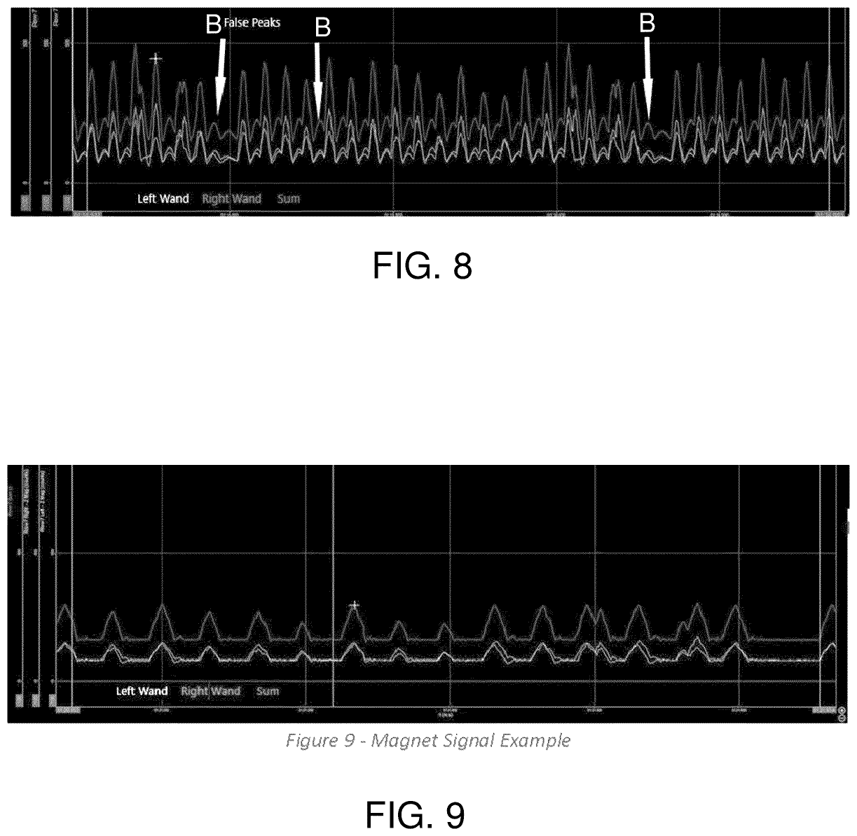

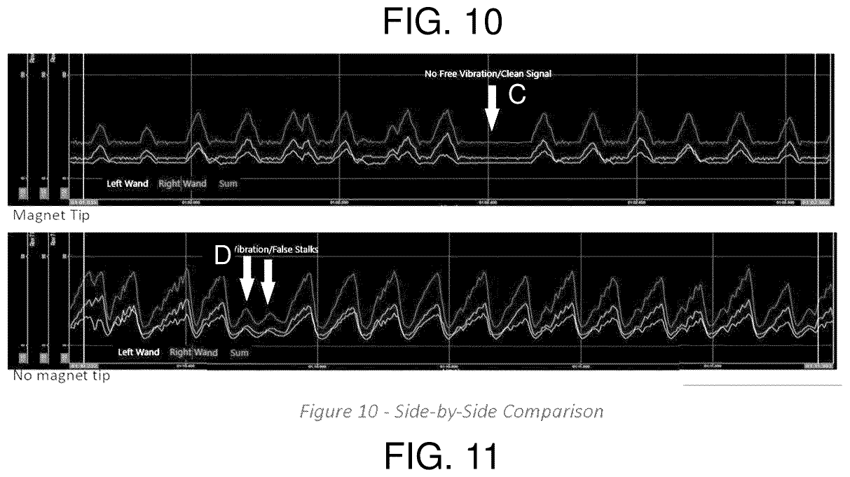

As would be appreciated, the wands 12A, 12B bouncing off stalks and noise from leaves and other debris can create peaks in the data that the algorithm identifies as false peaks in the signal that register as a stalk and over counting, as seen for example in FIG. 8 at B. These false peaks are caused by the free vibration of a wands 12A, 12B. The free vibration may be reduced with a material that is more damping and/or a stiffer hinge 18A, 18B. As disclosed herein, wands 12A, 12B including magnets 14A, 14B may also reduce signal noise as seen in FIGS. 9-11. FIG. 10 shows that between stalks (peaks) there is not vibration and a clean signal at C. FIG. 11 shows minimal vibration or false stalks at D.

The magnets 14A, 14B restrain the vibration of the wands 12A, 12B after a stalk has passed through the sensor 10. The damping effect of the magnets 14A, 14B reduces the need for a material or additional device to act as a damper. A material or device that is suitable for damping generally has a reduced cycle life because it absorbs the energy and produces heat which degrades the material. The benefits of the magnet 14A, 14B are a greater durability because free vibration is reduced without degradation. Because damping need not be as significant a factor in material selection, a more durable material can be selected for the wands 12A, 12B and hinges 18A, 18B.

When a wand 12A, 12B is cycled many times, it can lose its original shape (taking a set). Because the magnets 14A, 14B force the wands 12A, 12B to their home position, the effect of the set on the signal is reduced.

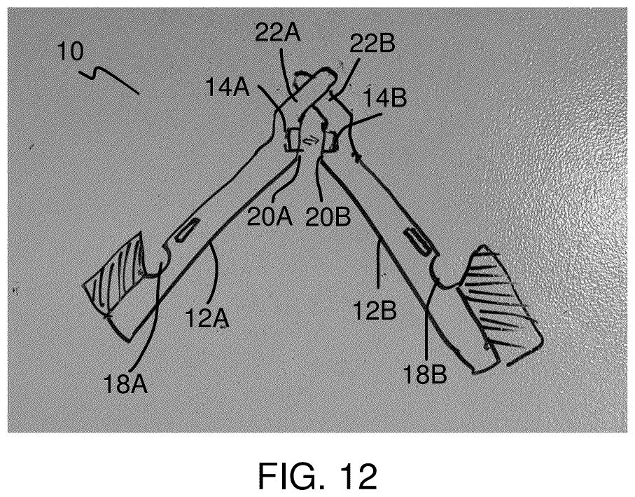

FIG. 12 shows a further implementation of a stalk sensor 10 where the wands 12A, 12B include an overlapping extension 22A, 22B at the tips and contacting surfaces 20A, 20B with magnets 14A, 14B to hold the wands 12A, 12B in the home position.

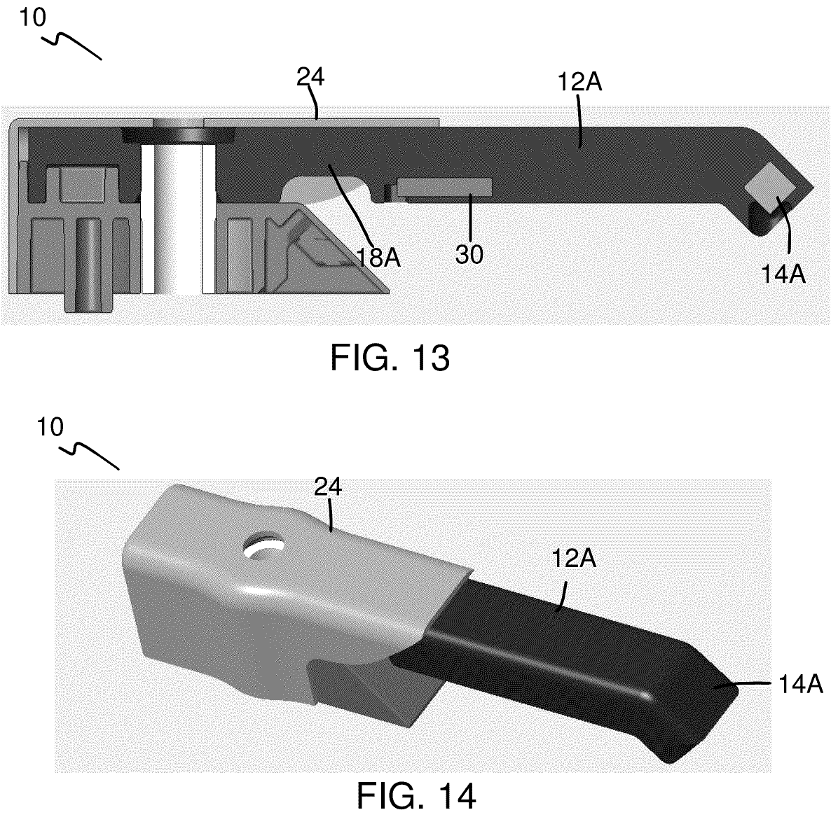

Turning to FIGS. 13-16, in various implementations a shield 24 that extends past the hinged portion 18 of the wand 12A could restrict the travel of the wand 12A and provide a physical home position and dampen the free vibration further creating a clean response signal form the sensor 10.

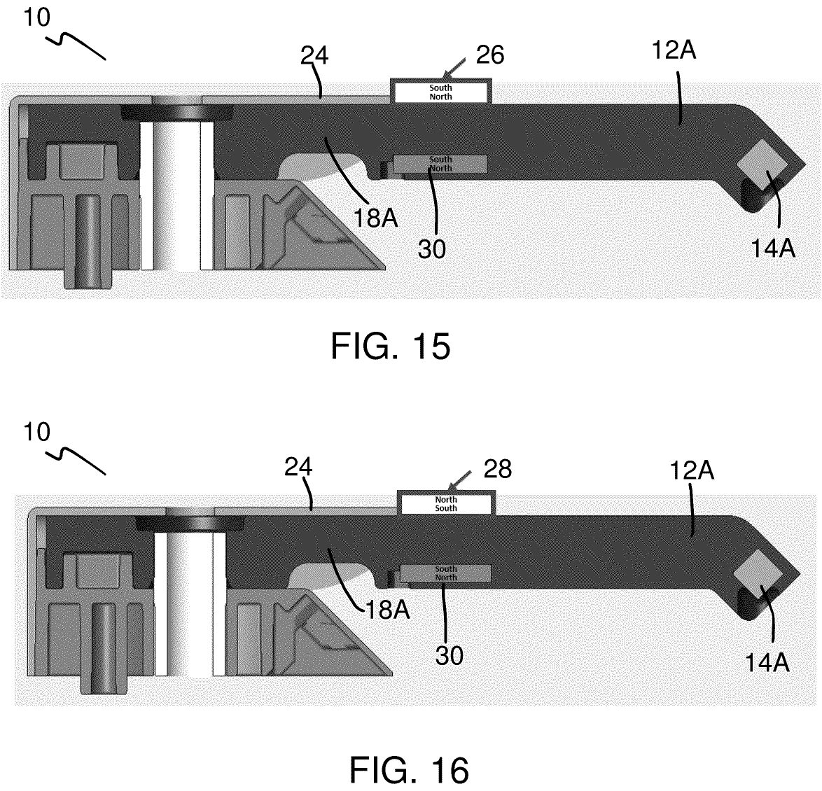

Various further implementations of the shield 24 include an attracting magnet 26 or ferrous piece of metal that attracts a sensing magnet 30 of the wand 12A to return the wand 12A to its home position as shown in FIG. 15. The sensing magnet 30 may be magnet that is read by a magnetometer reading the displacement of the wand 12A to detect stalks, as is described in the incorporated references. That is, as the wand 12A is displaced the sensing magnet 30 and the attracting magnet 26 are forced out of line (optionally vertical alignment) and once the stalk has passed through the sensor the wand 12A is returned to its home position with the assistance of the magnetic attraction between the sensing magnet 30 and the attracting magnet 26.

Alternatively, an opposing magnet 28 to the sensing magnet 30 could slow the wand 12A travel and dampen its motion as well, as shown in FIG. 16. That is, the opposing magnet 28 repels the sensing magnet 30 such that as the wand 12A is returning to its home position the opposing magnetic forces of the sensing magnet 30 and opposing magnet 28 must be overcome.

In implementations with an attracting magnet 26 or an opposing magnet 28 the wand 12A and senor 10 may optionally include a magnet 14A at the distal end of the wand 12A. Optionally, the magnet 14A at the distal end of the wand 12A may not be present in implementations having an attracting magnet 26 or opposing magnet 28.

In these and other implementations, the shield 24 is rigidly mounted in the same or similar was the sensor 10 is mounted to the harvester, row unit, or other implement as would be appreciated in the same or similar manner as the sensor 10 is mounted. Various mounting techniques and devices would be appreciated by those of skill in the art.

In various implementations, the hinge shield 24 provides a home position for the wands 12A, 12B whether used with the integrated magnet 26, 28 or piece of ferrous metal or without. In various implementations, the magnet 26, 28 or ferrous piece of metal could be added to the shield 24 in either orientation to dampen the vibration of the wand 12A, 12B and provide a signal with less free vibration. The hinge shield 24 also provides protection from abrasion for the hinge 18 region of the wand 12A, 12B to prevent early mechanical failures of the wand 12A, 12B.

The hinge shield 24 may bring the wands 12A, 12B to rest before the impact of the next stalk with the sensor. In various implementations the hinge shield 24 is an inexpensive part that provides a home position for the wands 12A, 12B and decrease free vibration. The shield 24 also protects the wands 12A, 12B and make the wands 12A, 12B and sensor 10 last longer in the field.

Although the disclosure has been described with references to various embodiments, persons skilled in the art will recognize that changes may be made in form and detail without departing from the spirit and scope of this disclosure.

What is claimed is:

1. A stalk sensor comprising:
(a) a first wand comprising a contact surface at a distal end;
(b) a first magnet disposed at a distal end of the first wand; and
(c) a second wand comprising a contact surface at a distal end,
wherein the first contact surface and the second contact surface are in contact when the stalk sensor is in a home position.

2. The stalk sensor of claim 1, further comprising a ferrous material disposed at a distal end of the second wand the ferrous material configured to attract the first magnet and thereby the first wand into contact with the second wand at the contact surfaces.

3. The stalk sensor of claim 2, wherein the first wand and second wand are self-aligning along the horizontal axis.

4. The stalk sensor of claim 1, further comprising a second magnet disposed at a distal end of the second wand, the magnet configured to attract the first magnet and thereby the first wand into contact with the second wand at the contact surfaces with the second wand.

5. The stalk sensor of claim 4, wherein a north pole of the first magnet is adjacent to the contact surface of the first wand and wherein a south pole of the second magnet is adjacent to the contact surface of the second wand.

6. The stalk sensor of claim 4, wherein north and south poles of the first magnet and second magnet are each adjacent to the contact surfaces.

7. The stalk sensor of claim 4, wherein poles of the first magnet and second magnet are oriented perpendicular to a direction of deflection of the first wand and second wand.

8. The stalk sensor of claim 7, wherein the wands are self-aligning on horizontal and vertical axes.

9. The stalk sensor of claim 1, further comprising a hinge shield configured to extend beyond a hinged portion of the first wand and restrict deflection of the first wand.

10. The stalk sensor of claim 9, further comprising a third magnet at a distal end of the hinge shield configured to attract a sensing magnet on the first wand and return the stalk sensor the home position.

11. The stalk sensor of claim 9, further comprising a third magnet at a distal end of the hinge shield configured to repel a sensing magnet on the first wand and dampen movement of the first wand toward the home position.

12. The stalk sensor of claim 9, wherein the hinge shield is configured to be mounted to a harvester row unit.

13. The stalk sensor of claim 1, wherein the first wand and second wand are configured to flex and separate the first contact surface and the second contact surface as a stalk passes through the stalk sensor.

14. The stalk sensor of claim 1, wherein the stalk sensor is configured to measure deflection of the first wand and second wand to count stalks as stalks pass through the sensor.

15. A sensing wand comprising a magnet at a distal end of the wand wherein a first pole of the magnet faces a contact surface at the distal end of the wand and a second pole of the magnet if faced away from the contact surface.

16. The sensing wand of claim 15, wherein a second sensing wand comprises a ferrous metal at a distal end of the second sensing wand configured to attract the magnet of the sensing wand.

17. The sensing wand of claim 15, further comprising a hinge shield configured to extend beyond a hinged portion of the sensing wand and restrict deflection of the sensing wand.

18. A sensing wand comprising a magnet at the distal end of the wand wherein north and south poles of the magnet are oriented perpendicular to a direction of deflection of the sensing wand.

19. The sensing wand of claim 18, further comprising a hinge shield configured to extend beyond a hinged portion of the sensing wand and restrict deflection of the sensing wand.

20. The sensing wand of claim 18, wherein the hinge shield is configured to be mounted to a harvester row unit.
